(19) United States
(12) Patent Application Publication
(10) Pub. No.: US 2011/0280378 A1
(43) Pub. Date: Nov. 17, 2011

(54) APPARATUS FOR HOLDING DIGITAL DENTAL X-RAY SENSOR AND METHOD OF MAKING SAME

(76) Inventors: Kent A. Feltz, Baxter, MN (US); Jeffrey J. Mattson, Baxter, MN (US)

(21) Appl. No.: 13/089,415

(22) Filed: Apr. 19, 2011

Related U.S. Application Data
(60) Provisional application No. 61/333,375, filed on May 11, 2010.

(51) Int. Cl. A61B 6/14; B23P 11/00

(52) U.S. Cl. 378/191; 29/428

(57) ABSTRACT

A dental x-ray sensor assembly includes a bite block used for digital imaging, wherein a surface of the bite block includes a deformable material that readily returns to an undeformed position after a biting force is removed therefrom.

### CROSS-REFERENCE TO RELATED APPLICATION

The present application is a non-provisional of, and claims priority to, U.S. Provisional Application 61/333,375 filed May 11, 2010, the disclosure of which is incorporated herein.

### BACKGROUND

Embodiments of the invention relate generally to dental x-ray sensor assembly, and more specifically to an apparatus for holding a dental x-ray sensor and a method of making same.

Traditionally, dental radiographs are made using an x-ray sensor that includes a film that is positioned within a patient's oral cavity and aligned to take an x-ray image of certain teeth using a film holder. The film is positioned such that x-rays directed toward the film first pass through the teeth and impinge upon the film. An image generated therefrom is based on an amount of x-ray attenuation that occurs, which depends on varying anatomical densities through which the x-rays pass.

In order to properly position and align the sensor with respect to the target teeth to be imaged, the film is held by a structure that includes an x-ray guide arm and a positioning ring. The guide arm extends generally in an orthogonal direction from a plane of the sensor, and the positioning ring is configured so that it may be translated along the generally orthogonal direction and locked in place on the guide arm for optimal use. The structure includes what is sometimes referred to as a styrofoam Stabe® (Stabe is a registered trademark of Dentsply International Inc. of Philadelphia, Pa.) or bite-piece and the film is structurally attached proximate thereto.

In use a new and unexposed film is placed on the film holder and placed within the patient's mouth proximate the tooth or teeth to be examined. The positioning ring is axially positioned and locked on the x-ray guide arm, providing a broad visual guide to a technician for alignment of an x-ray source. The patient is instructed to apply a mandibular force by biting on the styrofoam piece to hold the film in its desired location, and the technician applies x-rays toward the patient's mouth, and removes and processes the film to generate the image.

Advances in dental imaging have resulted in the use of digital devices instead of traditional film-based images, resulting in a number of improvements over the traditional system. For instance, film processing takes valuable time and includes the use of chemicals that must be purchased and then later disposed of The film itself is only used once and then stored in the patient's file, adding the cost of purchase and storage. Film-based images are not transferable digitally, thus the film must either be physically viewed in a viewer, or scanned and sent electronically. In fact there are a number of known benefits to the use of digital devices for obtaining dental images as compared to traditional film-based images, thus the overall industry trend is toward digital-based imaging.

A digital device includes a digital sensor that may include a charge coupled device or other known detection technologies that enable images to be generated without the need of a new film for each image. The digital sensor may be positioned within the oral cavity and used numerous times to obtain multiple subsequent sets of image data as the sensor is positioned and re-positioned for subsequent images. In order to obtain imaging data from the sensor, the sensor often includes a data cable that extends therefrom (some wireless devices are known but tend to not be used in the industry because of transmission issues, cost and risk of being inadvertently disposed of, and because of possible theft by patients, to name a few examples). The cable passes from the oral cavity and out of the patient's mouth between the patient's upper and lower jaw, and to a computing device that is used for image generation. Because the sensor is used numerous times in an imaging session, a styrofoam piece or Stabe® is typically not useable as a bite-block because styrofoam irreversibly distorts after each mandibular force sufficient to stabilize the sensor is applied. Also, in order to accommodate the digital sensor, a styrofoam piece or Stabe® typically is much larger than that used for traditional film and may not fit comfortably into the mouth of a patient. Therefore, bite-blocks for digital dental images are typically made of a high-durometer plastic to enable them to be smaller, and to be used and re-used multiple times for multiple image acquisitions during an imaging session.

However, the data cable for digital radiography can cause interference in the oral cavity and upon application of the mandibular force the digital sensor may be caused to move out of its desired position. That is, as a patient applies a lateral or biting force to the bite-block, the cable extending from the sensor may interfere with a patient's lips or teeth, as examples, causing motion of the sensor. Because the bite-block is typically fabricated from a hard plastic, and because of the cable interference, motion of the sensor may occur while imaging data is obtained, causing blurring in the image. In other words, the plastic bite-piece often moves out of position due to a lateral force caused by high durometer plastic properties, the mandibular vertical or lateral (bite) force, and the complex geometry (including the cable) of the bite piece. As a result, the image needs to be retaken, adding cost and time to the process, and adding radiation exposure to the patient.

The need exists, therefore, for a means to prevent the lateral force that often causes unintended bite-piece movement.

### BRIEF DESCRIPTION

The invention is a method and apparatus for improving dental digital radiographs.

According to an aspect of the invention, a dental x-ray sensor assembly includes a bite block used for digital imaging, wherein a surface of the bite block includes a deformable material that readily returns to an undeformed position after a biting force is removed therefrom.

According to another aspect of the invention, a method of fabricating a dental x-ray assembly includes providing a bite block that is couplable to a dental digital imaging sensor, the digital imaging sensor including a data cable attached thereto, and providing a resilient bite block material for at least one surface of the bite block.

According to yet another aspect of the invention, a dental x-ray sensor assembly includes a bite block couplable to a digital imaging dental sensor, and a cable connected to the dental x-ray sensor assembly and connectable to a computing device, wherein a surface of the bite block includes a resilient material that deforms when subjected to a stabilizing bite force and returns to an undeformed shape upon removal of the stabilizing bite force, and wherein the cable passes from an oral cavity of a patient when the digital imaging dental sensor is inserted into an oral cavity of a patient.

Various other features and advantages will be made apparent from the following detailed description and the drawings.

### BRIEF DESCRIPTION OF THE DRAWINGS

FIG. 1 is an illustration of a dental x-ray sensor assembly according to an embodiment of the invention.

FIGS. 2 and 3 illustrate a digital x-ray sensitive device inserted into a mouth or cavity in exemplary orientations.

FIGS. 4A-4C illustrate assembly steps of a bite block cover over a bite block corresponding to the assembly illustrated in FIG. 1.

FIG. 5 illustrates a single unit bite-block according to an embodiment of the invention.

FIG. 6 illustrates a bite block having a deformable material adhered thereto, according to the invention.

### DETAILED DESCRIPTION

Forms of the bite-piece are shown by the figures as described with respect to a digital dental x-ray sensor assembly. However, it is to be understood that the invention is not to be so limited, and that the invention is applicable to any device where a mandibular vertical force may be applied that causes an x-ray sensitive device to move after that device has been aligned with an x-ray source.

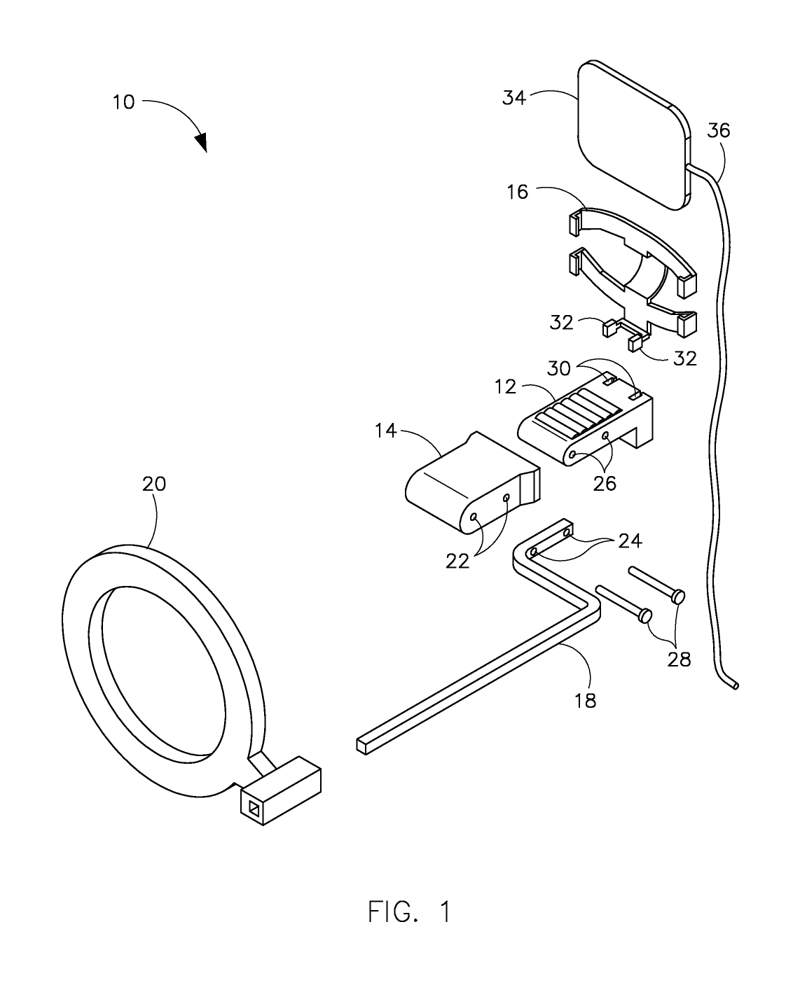

FIG. 1 is an illustration of a dental x-ray sensor assembly according to an embodiment of the invention. FIG. 1 illustrates an assembly 10 that includes a bite-piece or bite-block 12, a boot or bite-block cover 14, a sensor holder 16, and a support structure 18 coupled to an aiming ring 20. Bite-block 12 is fabricated from a hard, non-deformable plastic such as polycarbonate having a hardness of, for instance, 117 Rockwell R. However, bite-block 12 is not so limited and may be any material having sufficient structural integrity and hardness to support bite-block cover 14, such as a metal, a carbon composite, and the like. That is, bite-block 12 may be any structural material that may be inserted into a patient's mouth without adverse consequences (i.e., medical or food grade). Bite-block 12 includes apertures 22, support structure 18 includes apertures 24, and bite-block cover 14 includes apertures 26. Apertures 22, 24, and 26 are configured and positioned such that attachment mechanisms 28 may be passed therethrough in order to join or couple together bite-block 12, bite-block cover 14, and support structure 18.

Bite-block 12 includes passages 30, and sensor holder 16 includes mating prongs 32 that are configured to pass through passages 30 and engage therewith. Sensor holder 16 is configured to engage and hold a digital x-ray sensitive device 34 having a cable 36 attached thereto for passage of imaging data from digital x-ray sensitive device 34. Digital x-ray sensitive device 34 includes a film or a material that can convert x-rays to a digital signal and output the digital signal to a computing device (not shown). Assembly 10 is assembled by coupling sensor holder 16 to bite-block 12 via passages 30 and mating prongs 32. Bite-block cover 14 is positioned over bite-block 12, and support structure 18 having aiming ring 20 is coupled thereto via attachment mechanisms 28. Digital x-ray sensitive device 34 is coupled to sensor holder 16.

According to the invention, bite-block cover 14 is fabricated of a rubber or elastomeric material and may be, for instance, a medical or food grade rubber. In one example, bite-block cover 14 has a durometer between 25 and 100 Shore A and is selected to have a hardness that is less than that of bite-block 12. However, it is to be recognized that the invention is not limited to any specific durometer material, and any durometer material may be used that has a hardness lower than that of bite-block 12 and mitigates the lateral force that can cause unintended bite-piece movement. Rubber or elastomeric materials are selected for bite-block cover 14 such that bite-block cover 14 is resilient and deformable, thus will not plastically deform when the mandibular force is applied thereto. Instead, application of the mandibular force will cause bite-block cover 14 to elastically deform, leading to an increased friction between the teeth of the patient and assembly 10. As known in the art, typical coefficient-of-friction (COF) measurements may not apply to thermoset rubber compounds because of their ability to deform when a force is applied thereto. Instead, COF for such materials may be variable and dependent on a number of factors that include but are not limited to an amount of force applied during use. That is, a static COF may be increased in such a material by applying force and effectively increasing an amount of contact area thereon.

Therefore, compared to the hard surface of bite-block 12 (that may have a static COF below, for instance, 0.5) the use of a softer resilient material such as bite-block cover 14 will cause any motion of assembly 10 to tend to follow any relative lateral motion of the jaw of the patient, and not slip between the teeth, when the biting force is applied. Further, because bite-block cover 14 is resilient it will not plastically deform from application of the mandibular force, but will instead spring back to its shape that it was prior to when the mandibular force was applied and will be ready for a subsequent use. In other words, the use of bite-block cover 14 will enable multiple sequential dental images to be taken in a single patient by positioning and repositioning assembly 10 within the oral cavity, without the need to change out, for instance, styrofoam pieces as is done in conventional films. Further, because of the elastic deformation of bite-block cover 14, assembly 10 may be correctly positioned for each subsequent image and assembly 10 will not move out of position due to the mandibular vertical force.

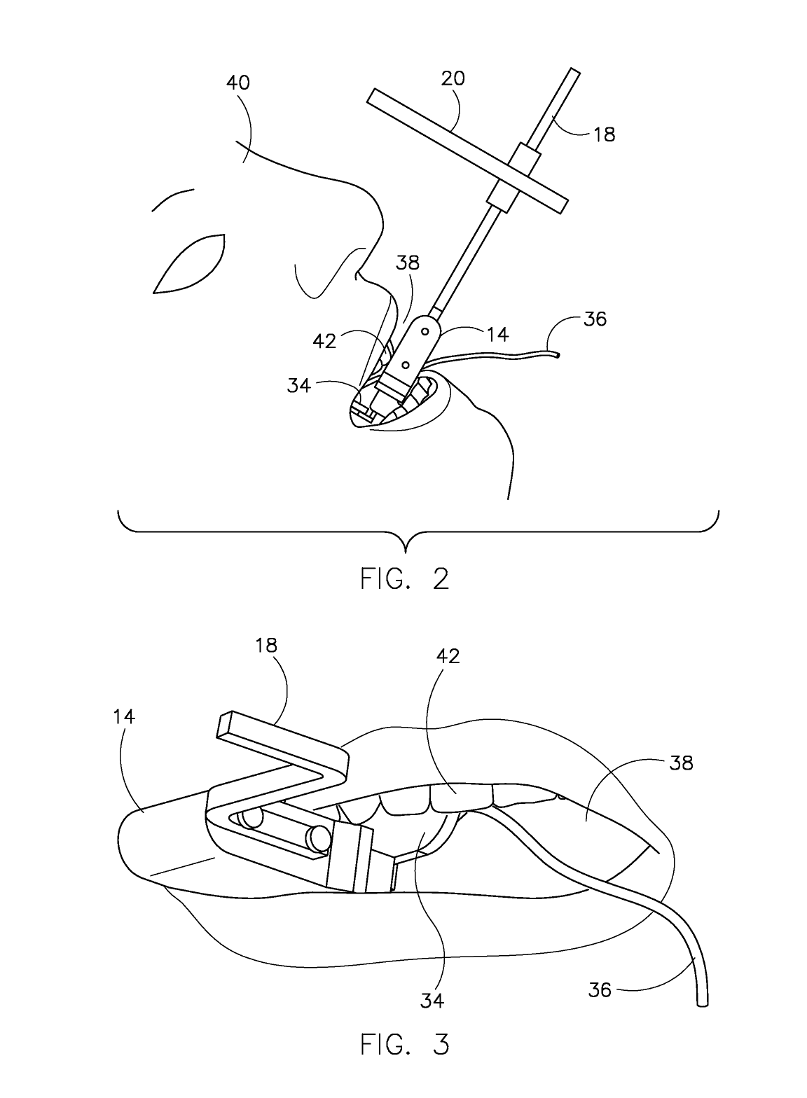

Referring now to FIGS. 2 and 3, digital x-ray sensitive device 34 is inserted into a mouth or oral cavity 38 of a patient 40, and patient 40 is instructed to apply a mandibular or bite force against bite-block cover 14 that is sufficient enough to stabilize bite-block 12 (positioned inside of bite-block cover 14, according to this embodiment) for digital imaging. That is, despite the use of cable 36, according to the invention a technician can hold and position assembly 10 while the mandibular force is applied. Because of the softness of bite-block cover 14, teeth 42 deform bite-block cover 14, enabling a solid ‘grip’ thereon, and enabling assembly 10 to remain in a desired position until the technician aligns and energizes an x-ray source (not shown) relative to aiming ring 20. FIGS. 2 and 3 illustrate two positions for placement of x-ray sensitive device 34 within mouth or oral cavity 38. However, FIGS. 2 and 3 illustrate only representative positions of x-ray sensitive device 34 and assembly 10 in general, and one skilled in the art will recognize that many orientations of x-ray sensitive device 34 and assembly 10, relative to teeth 42, may be accomplished according to the invention. Bite-block cover 14 thus resolves concerns of motion of assembly 10 by providing a bite-block cover 14 that transmits the mandibular force vertically and thereby mitigates the lateral force causing unintended bite-piece movement.

The invention described herein is not limited to the embodiment illustrated in FIG. 1 and in use in FIGS. 2-3, but is applicable to any dental x-ray sensor assembly where a bite piece is inserted into a jaw of a patient having an x-ray sensitive device coupled thereto.

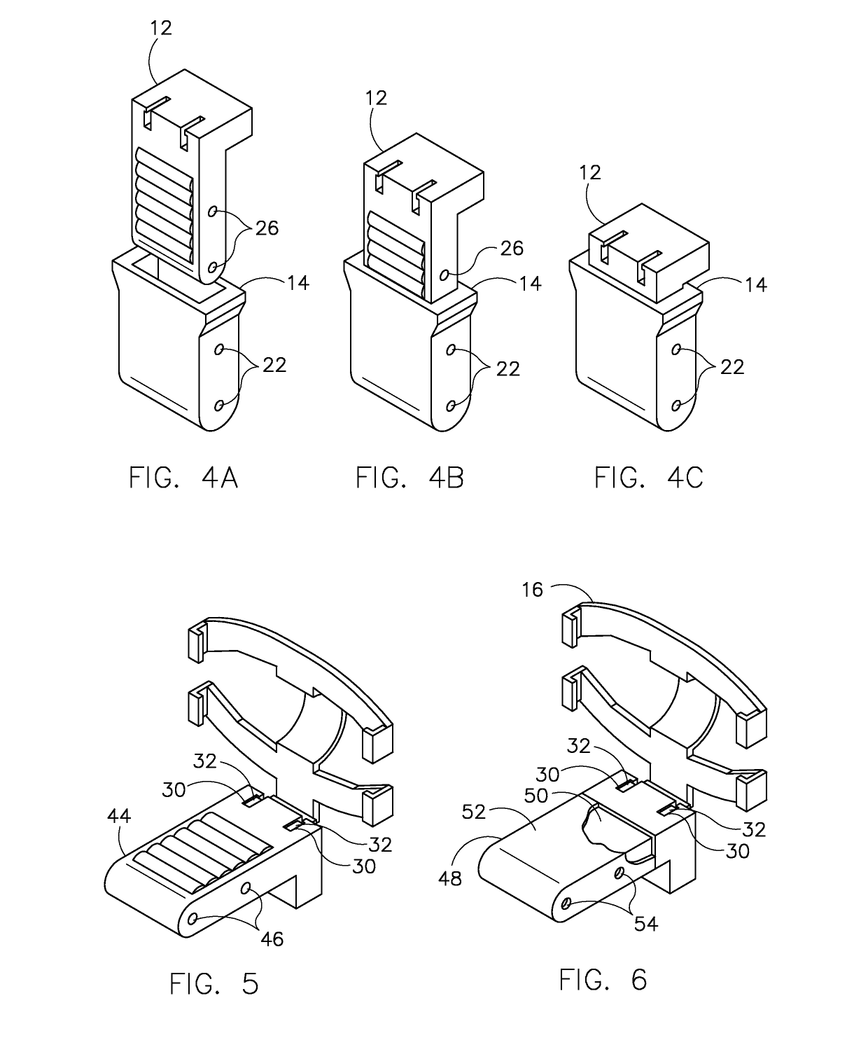

FIG. 4 illustrates assembly steps of bite-block 12 and bite-block cover 14 of FIG. 1. FIG. 4A shows the two components 12, 14 just prior to insertion of bite-block 12 into bite-block cover 14, FIG. 4B shows partial insertion, and FIG. 4C shows components 12, 14 with respective apertures 22, 26. Thus, as described, bite-block 12 is formed having a first hardness, and positionable bite-block cover 14, having a second hardness that is less than the first hardness, has bite-block 12 inserted thereinto to cover a portion of bite-block 12.

According to another embodiment of the invention, bite-block 12 and bite-block cover 14 are formed of a single material. Referring to FIG. 5, a single unit bite-block 44 is formed of a low durometer material and is fabricated of a rubber or elastomeric material and may be, for instance, a medical or food grade rubber. Single unit bite-block 44 includes passages 30 such that prongs such as mating prongs 32 of sensor holder 16 may be inserted thereinto. In one example, single unit bite-block 44 has a durometer between 25 and 100 Shore A and is selected based on its resilience to deformation, and also based on its ability to retain mating prongs 32 of sensor holder 16. Single unit bite-block 44 also includes apertures 46 in order to be assembled with other components in a similar fashion as shown in assembly 10 of FIG. 1. That is, in lieu of using two components 12, 14 as shown in FIG. 1, a bite block such as single unit bite-block 44 may be entirely formed of a deformable or resilient material.

According to still another embodiment of the invention, bite-block 12 and bite-block cover 14 may be formed using a skeletal underlying material having a deformable material adhered thereto. Referring to FIG. 6, a bite-block 48 includes a non-deformable skeletal underlying material 50 and a deformable material 52 adhered thereto. Bite-block 48 includes apertures 54 in order to be assembled with other components in a similar fashion as shown in assembly 10 of FIG. 1. That is, in lieu of using two components 12, 14 as shown in FIG. 1, bite-block 48 may be formed of a deformable or resilient material 52 adhered to skeletal underlying material 50. According to the invention, skeletal underlying material 50 may be formed of a hard non-deformable plastic, such as a polycarbonate. Deformable material 52 is formed of a low-durometer material and is fabricated of a rubber or elastomeric material and may be, for instance, a medical or food grade rubber. Bite-block 48 includes passages 30 such that prongs such as mating prongs 32 of sensor holder 16 may be inserted thereinto. In one example, deformable material 52 has a durometer between 25 and 100 Shore A and is selected based on its resilience to deformation.

Regarding the embodiments of FIGS. 5 and 6, any durometer material may be used for single unit bite-block 44 or for deformable material 52 that mitigates the lateral force that can cause unintended bite-piece movement. Rubber or elastomeric materials are selected such that single unit bite-block 44 or deformable material 52 is resilient and deformable, thus will not plastically deform when the mandibular force is applied thereto. Instead, application of the mandibular force will cause single unit bite-block 44 or deformable material 52 to elastically deform, leading to an increased friction between the teeth of the patient and assembly 10. That is, the use of a resilient material will cause any motion of assembly 10 to tend to follow any relative lateral motion of the jaw of the patient, and not slip between the teeth, when the biting force is applied. Further, because the material is resilient it will not plastically deform from application of the mandibular force, but will instead spring back to its shape that it was prior to when the mandibular force was applied, and will be ready for a subsequent use. In other words, embodiments of the invention will enable multiple sequential dental images to be taken in a single patient by positioning and repositioning assembly 10 within the oral cavity, without the need to change out, for instance, styrofoam pieces as is done in conventional films. Further, because of the elastic deformation, assembly 10 may be correctly positioned for each subsequent image and assembly 10 will not move out of position due to the mandibular vertical force.

Referring to assembly 10 of FIG. 1, one skilled in the art will recognize that not all such assemblies include, for instance, a structure such as support structure 18 or an aiming ring such as aiming ring 20. Thus, a bite piece may be fabricated and covered with a bite piece cover, the bite piece having an x-ray sensitive device attached directly thereto, or coupled thereto via a x-ray device holder. Other examples may include an aiming device other than a ring illustrated by aiming ring 20.

This invention is applicable to any device, its manufacture, and method of use, where a mandibular vertical force may be applied that causes an x-ray sensitive device to move after that device has been aligned with an x-ray source. In one example, material used to which the mandibular force is applied (bite-block cover 14, single unit bite-block 44, and deformable material 52) is Santoprene 8271-65, which is a thermoplastic vulcanizate. Santoprene is a registered trademark of ExxonMobil Corporation, located in Irving, Tex. In general, Santoprene products have a static COF that is greater than 0.5, and such products typically increase in static COF as more force is applied thereto. However, material for bite-block cover 14, single unit bite-block 44, and deformable material 52 is not so limited and may be any deformable or resilient material, that returns to an undeformed position or shape after removal of a bite force) having an increased COF when a force is applied thereto.

Further, one skilled in the art will recognize that the material selection to which the mandibular force is applied may not be limited to the durometer of 25-100 Shore A, and that any material may be selected in order to reduce or eliminate motion of an x-ray sensitive device after that device has been aligned with an x-ray source.

The bite piece cover described herein is intended to prevent a mandibular lateral force from causing movement of an x-ray bite piece sensor holder. It is an intention of the invention to include a material that prevents a mandibular lateral force from causing motion of assembly 10 by including a low durometer bite piece cover that focuses the mandibular force vertically and mitigates the lateral force. It is another intention of the invention to provide a bite piece cover that can accommodate a variety of sensor holders and not only sensor holder 16 having mating prongs 32.

According to an embodiment of the invention, a dental x-ray sensor assembly includes a bite block used for digital imaging, wherein a surface of the bite block includes a deformable material that readily returns to an undeformed position after a biting force is removed therefrom.

According to another embodiment of the invention, a method of fabricating a dental x-ray assembly includes providing a bite block that is couplable to a dental digital imaging sensor, the digital imaging sensor including a data cable attached thereto, and providing a resilient bite block material for at least one surface of the bite block.

According to yet another embodiment of the invention, a dental x-ray sensor assembly includes a bite block couplable to a digital imaging dental sensor, and a cable connected to the dental x-ray sensor assembly and connectable to a computing device, wherein a surface of the bite block includes a resilient material that deforms when subjected to a stabilizing bite force and returns to an undeformed shape upon removal of the stabilizing bite force, and wherein the cable passes from an oral cavity of a patient when the digital imaging dental sensor is inserted into an oral cavity of a patient.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A dental x-ray sensor assembly comprising:
a bite block used for digital imaging, wherein a surface of the bite block comprises a deformable material that readily returns to an undeformed position after a biting force is removed therefrom.

2. The assembly of claim 1 comprising:
a digital x-ray sensor having a cable extending therefrom;
wherein the dental x-ray sensor assembly is configured to convert x-rays to a digital signal, and output the digital signal to a computing device.

3. The assembly of claim 1 wherein the biting force is substantial enough to stabilize the bite block for digital imaging.

4. The assembly of claim 1 wherein the bite block comprises a skeletal underlying material having the deformable material adhered to the skeletal underlying material, and wherein the skeletal underlying material has a hardness that is greater than a hardness of the deformable material.

5. The assembly of claim 4 wherein the skeletal underlying material is a non-deformable plastic.

6. The assembly of claim 1 wherein the bite block is entirely comprised of the deformable material.

7. The assembly of claim 1 wherein the bite block comprises:
a block having a first hardness; and
a positionable boot positioned to cover at least a portion of the block, the positionable boot comprising the deformable material and having a second hardness that is less than the first hardness.

8. The assembly of claim 1 wherein the deformable material has a friction coefficient greater than a friction coefficient of polycarbonate.

9. The assembly of claim 1 wherein the deformable material comprises one of a medical and a food grade rubber.

10. The assembly of claim 1 wherein the deformable material comprises a material having a durometer between 25 and 100 Shore A.

11. The assembly of claim 1 wherein a friction coefficient of the surface of the bite block is greater than 0.5.

12. A method of fabricating a dental x-ray assembly comprising:
providing a bite block that is couplable to a dental digital imaging sensor, the digital imaging sensor comprising a data cable attached thereto; and
providing a resilient bite block material for at least one surface of the bite block.

13. The method of claim 12 wherein providing the resilient bite block material comprises providing the resilient bite block material that is reusable for a subsequent use and undeformed from a prior use.

14. The method of claim 12 wherein providing the bite block comprises providing a skeletal material, and wherein providing the resilient bite block material comprises providing the resilient bite block material as a skin that is adhered to the skeletal material.

15. The method of claim 12 wherein a removable bite block cover comprises the resilient bite block material, and further comprising covering the bite block with the bite block cover.

16. The method of claim 12 wherein providing the bite block comprises providing the bite block that is comprised entirely of the resilient bite block material.

17. A dental x-ray sensor assembly comprising:
a bite block couplable to a digital imaging dental sensor; and
a cable connected to the dental x-ray sensor assembly and connectable to a computing device;
wherein a surface of the bite block comprises a resilient material that deforms when subjected to a stabilizing bite force and returns to an undeformed shape upon removal of the stabilizing bite force; and
wherein the cable passes from an oral cavity of a patient when the digital imaging dental sensor is inserted into an oral cavity of a patient.

18. The assembly of claim 17 wherein the bite block comprises a structural underlying material having the resilient material adhered to the structural underlying material, and wherein the structural underlying material has a hardness that is greater than a hardness of the resilient material.

19. The assembly of claim 17 wherein the bite block is comprised entirely of the resilient material.

20. The assembly of claim 17 wherein the bite block comprises:
a structural component having a first hardness; and
a positionable boot positioned over the structural component, the positionable boot comprising the resilient material and having a second hardness that is less than the first hardness.
